(19) United States
(12) Patent Application Publication
(10) Pub. No.: US 2010/0145128 A1
(43) Pub. Date: Jun. 10, 2010

(54) C7 ISOMERISATION WITH REACTIVE DISTILLATION

(76) Inventor: Sven Ivar Hommeltoft, Pleasant Hill, CA (US)

Correspondence Address: DICKSTEIN SHAPIRO LLP, 1825 EYE STREET NW, Washington, DC, 20006-5403, US

(21) Appl. No.: 12/090,466

(22) PCT Filed: Nov. 13, 2006
(86) PCT No.: PCT/EP06/10850

§ 371 Date: Apr. 16, 2008

(30) Foreign Application Priority Data
Nov. 22, 2005 (PA) PA 2005-01638

(51) Int. Cl. C07C 5/13

(52) U.S. Cl. 585/737

(57) ABSTRACT

Process for isomerising a hydrocarbon feed containing at least C₇ hydrocarbons, comprising steps of (a) in a separation column separating the feed into a heavy fraction comprising hydrocarbons having higher boiling point than n-heptane, an intermediate fraction being rich in n-heptane and/or mono-branched iso-heptanes and a light fraction being rich in multi-branched iso-heptanes; (b) withdrawing continuously from the separation column a portion of the intermediate fraction being rich in n-heptane and/or mono-branched iso-heptanes; (c) introducing the withdrawn portion into an isomerisation reactor and isomerising at isomerisation conditions the portion in presence of an isomerisation catalyst and a gas stream being rich in hydrogen; (d) withdrawing from the isomerisation reactor an isomerised effluent stream being enriched in multi-branched iso-heptanes together with cracked hydrocarbons and hydrogen; (e) purging the cracked hydrocarbons and hydrogen from the isomerised effluent to obtain a stabilised reactor effluent; (f) recycling and introducing the stabilised reactor effluent into the separation column; and (g) withdrawing from the separation column a top product being rich in multi-branched C₇ isomers.

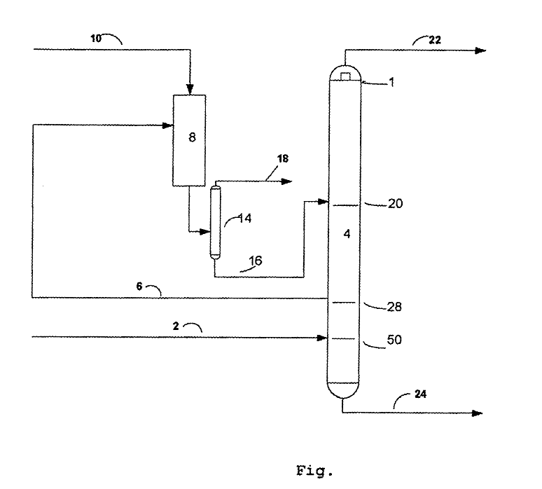

### FIELD OF THE INVENTION

The present invention is directed towards an isomerisation of a paraffinic hydrocarbon feedstock. In particular, the invention concerns isomerisation of a C₇ hydrocarbon cut by combined fractionation and catalytic isomerisation. The process comprises separation of the feedstock into different fractions in a fractionator, wherein at least one fraction is rich in C₇ hydrocarbons, isomerisation of the fraction in a separate isomerisation unit in presence of an isomerisation catalyst and recycling of the isomerised fraction back to the fractionator for the production of multi-branched paraffins.

### BACKGROUND OF THE INVENTION

There is an increasing need to find substitutes for previously used octane busters in gasoline such as environmental and health hazardous aromatic compounds. Multi-branched paraffins are ideal gasoline-blending components possessing high octane numbers and low or no hazardous properties. It is therefore an incentive to develop processes for increasing the octane number of paraffinic hydrocarbons by isomerisation of suitable normal paraffin fractions, such as low octane C₄ to C₁₂ cuts. While C₅/C₆ paraffin isomerisation is a common refinery process, utilisation of C₇⁺ fractions meets significant difficulties given by the usually high degree of cracking those fractions to gas.

Paraffin isomerisation is equilibrium limited reaction and for higher fractions including C₇ hydrocarbons, isomerisation is accompanied by cracking reactions. The relative cracking selectivity increases as isomerisation conversion increases, because the isomerisation reaction rate decreases as the equilibrium is approached, whereas cracking is an irreversible reaction and not influenced by equilibrium conditions. A further problem with isomerisation of higher paraffinic hydrocarbons is cracking of the isomerised paraffin products, which are more readily cracked than their corresponding normal-paraffins.

For the equilibrium-limited isomerisation reaction conversion can be increased by removing the products continuously during reaction by performing the reaction under distillation conditions using reactive distillation.

Reactive distillation in the isomerisation of hydrocarbons is known in the art.

Thus, U.S. Pat. Nos. 5,948,948, 6,054,630 and 6,084,141 describe paraffin isomerisation employing a reactive distillation process with a distillation zone associated with a reaction zone, which is at least in part internal to said distillation zone and comprises one or more catalytic beds in which the feed is transformed in the presence of a catalyst and hydrogen.

As known to those skilled in the art, hydrogen flow through the isomerisation catalyst bed has to be maintained at a sufficient partial pressure in order to prevent cooking of the catalyst and to optimise efficiency of the catalyst.

This limits the usefulness of the above known reactive distillation in which the isomerisation is be performed partly internal the distillation column since hydrogen being present in the catalyst is continuously removed together with the liquid flow from the catalyst through the top of the column.

A further disadvantage of reactive distillation, when employed in catalytic isomerisation is the presence of cracked products being in gas form and hydrogen in the distillation column. Presence of gaseous compounds decreases distillation efficiency. Consequently, the number of condensation trays in such a column must be increased in order to maintain reasonable separation of the different product fractions.

Still a disadvantage of the above known processes is reintroduction of isomerised products from the internal and external reaction zones to a level in the separation column being in close proximity to the draw-off tray. As already mentioned above, isomerised multi-branched paraffins are readily cracked and reintroduction of those compounds at substantially the same level from which the fraction to be isomerised is drawn-off will result in increased cracking of isomerate.

### SUMMARY OF THE INVENTION

The general object of this invention is to provide a process for the isomerisation of a hydrocarbon feed being rich in C₇ hydrocarbons without the above discussed disadvantages.

The object of the invention can be fulfilled, when performing the isomerisation process in an external isomerisation reactor with an intermediate fraction being rich in n-heptane and mono-branched heptanes being withdrawn from the separation column and purging hydrogen and cracked products being formed during isomerisation prior to reintroducing the isomerate into the separation column.

Thus, the isomerisation process of this invention comprises steps of

(a) in a separation column separating a hydrocarbon feed containing at least C₇ hydrocarbons into a heavy fraction with hydrocarbons having higher boiling point than n-heptane, an intermediate fraction being rich in n-heptane and/or mono-branched iso-heptanes and a light fraction being rich in multi-branched iso-heptanes;

(b) withdrawing continuously from the separation column at least a portion of the intermediate fraction being rich in n-heptane and/or mono-branched iso-heptanes;

(c) introducing the withdrawn portion into an isomerisation reactor and isomerising the portion at isomerisation conditions in presence of an isomerisation catalyst and a gas stream being rich in hydrogen;

(d) withdrawing from the isomerisation reactor an isomerised effluent stream being enriched in multi-branched isoheptanes together with cracked hydrocarbons and hydrogen;

(e) purging the cracked hydrocarbons and hydrogen from the isomerised effluent to obtain a stabilised reactor effluent;

(f) recycling the stabilised reactor effluent to the separation column; and

(g) withdrawing from the separation column a top product being rich in multi-branched C₇ isomers.

A typical hydrocarbon stream for use in the inventive process as feed to the separation column is rich in n-heptane and iso-heptanes. The feed can additionally contain other C₇ hydrocarbons such as C₇ naphthenes, toluene and C₇ olefins. Additionally, the feed may contain substantial amounts of C₆ and heavier hydrocarbons.

The hydrocarbon feed is introduced into the separation column at a level below or above the draw-off level to the isomerisation reactor depending on the composition of the feed. In cases where the feed stream is rich in toluene and/or C₈⁺ hydrocarbons it may be advantageous to introduce the process feed into the separation column at a level below the level at which the reactor feed for the isomerisation is withdrawn from the column. With feed compositions being lean or do not contain toluene and heavier hydrocarbons, it is preferred to introduce the feed into the column at a level above the draw-off level.

In accordance with the general principle of the invention, the hydrocarbon fraction to be isomerised is continuously drawn-off from a given level in the separation column with an intermediate liquid fraction being rich in n-heptane and/or mono-branched iso-heptanes, i.e. methyl hexanes and passed to an external isomerisation reactor.

Isomerisation of n-heptane and mono-branched iso-heptanes occurs at substantially known methods in presence of an isomerisation catalyst and hydrogen being introduced into the reactor by means of a stream being rich in hydrogen, preferably at least 50 mole %. The hydrogen stream may further contain light hydrocarbons such as for instance methane, ethane, propane or butane without adversely affecting the isomerisation reactions. Further typical operation conditions are temperatures between 100° C. and 300° C., total pressures varying between 1 and 100 bars and liquid space velocities (LHSV) between 0.1 and 30 h⁻¹. Preferred conditions are temperatures between 130° C. and 250° C., LHSV between 0.5 and 5h⁻¹ and an operation pressure between 5 and 50 bars. Preferably, the partial hydrogen pressure in the reactor is maintained at a between 5 and 50 bar.

Suitable catalysts for the isomerisation of C₇ hydrocarbons are any of isomerisation catalyst known to those skilled in the art. Examples of useful catalysts include zeolites and alumina based catalysts, and sulphated or tungstated zirconia catalysts combined with a hydrogenation catalyst component as disclosed in EP 1402947 A, which by reference thereto is incorporated herein.

When employing the above isomerisation conditions, the effluent from the isomerisation reactor will be at lower boiling point range than that of the fraction being withdrawn from the separation column for isomerisation and will be enriched in low boiling high octane multi-branched iso-heptanes. Thus, the isomerisation product contains 2,2,3-trimethylbutane (223TMB), 2,2-dimethylpentane (22DMP), 2,4-dimethyl pentane (24DMP) and 3,3-dimethylpentane (33DMP).

As already discussed above isomerisation reaction is an equilibrium reaction, which limits the concentration of the multi-branched isomers. The product contents further hydrogen and minor amounts of other heptane isomers and lighter hydrocarbons (C₄-C₆), which may be present in the isomerisation process feed or may be formed in the isomerisation reactor by cracking. These by-products are in the gas form and have a negative impact on the separation efficiency, if reintroduced into the separation column, as already discussed in the above description.

It is, thus, one of the characteristic features of the invention to remove gaseous by-products from the isomerised product prior to reintroducing the product into the separation column.

Methods for removal of gaseous compounds from a liquid per se known in the art and are typically based on phase separation, flash distillation or fractionation. In the process of this invention the isomerised product is in one embodiment subjected to separation being carried out either external or internal in the isomerisation reactor. The gaseous phase is purged and the remaining stabilised liquid effluent of isomerised products is passed to the separation column. In another embodiment removal of gaseous by-products is obtained by distillation in an external fractionator.

By either embodiment a stabilised liquid effluent is obtained containing the above mentioned multi-branched heptanes. The boiling point range of the effluent is lower than the boiling point range of the fraction having been drawn-off from the separation column as isomerisation feed.

Consequently, it will be preferred to reintroduce the isomerised product at a lower boiling point level, i.e. on a tray closer to the top tray in the separation column for further separation of the multi-branched isomers from non-converted n-heptane and mono-branched heptanes being present in the isomerised product. When reintroducing the product at a lower boiling point level closer to the top of the separation column, fewer amounts of the multi-branched hydrocarbons are recycled to the isomerisation reactor together with the hydrocarbon fraction to be isomerised. As a result, undesired cracking of the multi-branched heptanes in the isomerisation reactor is reduced.

The overhead being withdrawn at top of the column is the rich in the above mentioned multi-branched heptanes having a research octane number (RON) of between 80 and 120 and being a valuable product for incorporation into the gasoline pool.

The bottom product of the process mainly comprises toluene and naphtenes together with C₈ and heavier hydrocarbons with a boiling point higher than n-heptanes.

### DETAILED DESCRIPTION AND ILLUSTRATION OF THE INVENTION

In the following the invention will be explained in greater detail by reference to drawings, in which the sole Figure shows a simplified flow sheet of a specific embodiment of the invention.

A process feed stream 2 of C₆-C₉ naphtha with about 50% by volume of C₇ hydrocarbons is introduced into separation column 4. The stream is introduced at a point below the draw-off point for withdrawal of an intermediate fraction 6, because of the high content of C₈ and heavier hydrocarbons in the feed stream. In this embodiment of the invention, separation column 4 contains 68 theoretical trays (not shown) being numbered from the top the column. Feed stream 2 is introduced onto tray 50 and intermediate fraction 6 to be isomerised is withdrawn from tray 28 and passed to isomerisation reactor 8. A hydrogen rich stream is introduced into reactor 8 through line 10. An isomerised effluent stream 12 from reactor is stabilised by fractionated distillation in fractionator 14 into a liquid phase being passed to separation column 4 in line 16. The gaseous phase containing hydrogen and LPG is purged from separator 14 via line 18. The stabilised liquid effluent is reintroduced into separator 4 onto theoretical tray 20. The final isomerate product 22 is withdrawn from theoretical tray 1 and a bottom product 24 from theoretical tray 68.

The composition of the various streams and effluents in the above embodiment of the invention is summarised in the Table below.

TABLE

| | Process feed | Heavy fraction | Intermediate fraction | Stabilised reactor | Isomerate |
|---|---|---|---|---|---|
| | (2) to | (24) from | (6) from tray 28 | effluent (16) | product (22) |
| Component | tray 50 | tray 68 | to reactor (8) | to tray 20 | from tray 1 |
| Hydrogen | 0 | 0 | 0 | 0 | 0 |
| C₃ | 0 | 0 | 0 | 0 | 0 |
| C₄ | 0 | 0 | 0 | 0 | 0 |
| C₅ | 0 | 0 | 0 | 0 | 0 |
| C₆ | 0.21 | 0 | 0.13 | 0.12 | 0.19 |
| 223TMB | 0.01 | 0 | 0.03 | 0.05 | 0.03 |
| 22DMP | 0.04 | 0 | 0.22 | 0.56 | 0.38 |
| 24DMP | 0.04 | 0 | 0.25 | 0.55 | 0.34 |
| 33DMP | 0.04 | 0 | 0.36 | 0.36 | 0.04 |
| 23DMP | 0.09 | 0 | 0.73 | 0.65 | 0.01 |
| 3ETP¹ | 0 | 0 | 0.25 | 0.25 | 0 |
| 2MHEX² | 0.26 | 0 | 2.5 | 2.29 | 0.05 |
| 3MHEX³ | 0.31 | 0 | 2.36 | 2.06 | 0.02 |
| n-heptane | 0.25 | 0.12 | 1.4 | 1.27 | 0 |
| C₇-naphthenes | 0.12 | 0.11 | 0.33 | 0.32 | 0 |
| toluene | 0.01 | 0.01 | 0 | 0 | 0 |
| C₈⁺ | 0.91 | 0.91 | 0 | 0 | 0 |
| C6− | 0.21 | | | | |
| C7 | 1.17 | | | | |
| C8+ | 0.91 | | | | |

¹3-ethyl pentane,
²2-methyl hexane,
³3-methyl hexane

1. Process for isomerising a hydrocarbon feed containing at least C7 hydrocarbons, comprising steps of
a) in a separation column separating the feed into a heavy fraction comprising hydrocarbons having higher boiling point than n-heptane, an intermediate fraction being rich in n-heptane and/or mono-branched iso-heptanes and a light fraction being rich in multi-branched iso-heptanes;
b) withdrawing continuously from the separation column a portion of the intermediate fraction being rich in n-heptane and/or mono-branched iso-heptanes;
c) introducing the withdrawn portion into a isomerisation reactor and isomerising at isomerisation conditions the portion in presence of an isomerisation catalyst and a gas stream being rich in hydrogen;
d) withdrawing from the isomerisation reactor an isomerisised effluent stream being enriched in multi-branched iso-heptanes together with cracked hydrocarbons and hydrogen;
e) purging the cracked hydrocarbons and hydrogen from the isomerisised effluent to obtain a stabilised reactor effluent;
f) recycling and introducing the stabilised reactor effluent into the separation column; and
g) withdrawing from the separation column a top product being rich in multi-branched C7 isomers, wherein the stabilised reactor effluent in step (f) is introduced into the separation column at a level having a lower boiling point range than the boiling point range of the fraction being withdrawn from the separation column in step (b).

2. The process of claim 1, wherein the portion of the intermediate fraction is withdrawn from the separation column in step (b) at a level below or above the level at which the hydrocarbon feed is introduced into the separation column.

3. The process of claim 1, wherein the isomerisation conditions in step (c) comprise a temperature of between 100 and 300° C., a total reactor pressure of between 1 and 100 bar and a partial pressure of hydrogen between 2 and 50 bar.

4. The process of claim 1, wherein the purging of cracked hydrocarbons and hydrogen in step (e) is performed internally and/or externally to the isomerisation reactor.
